(19) United States
(12) Patent Application Publication
(10) Pub. No.: US 2022/0298206 A1
(43) Pub. Date: Sep. 22, 2022

(54) An improved process for the preparation of Etelcalcetide Hydrochloride

(71) Applicant: Auro Peptides Ltd, Hyderabad (IN)

(72) Inventors: Abdul Shafee Mohammed, Hyderabad (IN); Bharti Deshmukh, Hyderabad (IN); Vivekananda Reddy Goli, Hyderabad (IN); Riyaz Shaik, Hyderabad (IN); Narayan Ratan Thombare, Hyderabad (IN); Nagana Goud Agasaladinni, Hyderabad (IN)

(21) Appl. No.: 17/638,181

(22) PCT Filed: Aug. 25, 2020
(86) PCT No.: PCT/IB2020/057916

§ 371 Date: Feb. 24, 2022

(30) Foreign Application Priority Data
Aug. 26, 2019 (IN) 201941034285

(51) Int. Cl. C07K 7/06

(52) U.S. Cl. CPC C07K 7/06

(57) ABSTRACT

The present invention relates to an improved process for the preparation of Etelcalcetide hydrochloride of Formula (I): The present invention also provides a process for preparing Etelcalcetide acetate of Formula (VI), which is an intermediate of Etelcalcetide hydrochloride: Formula (VI)

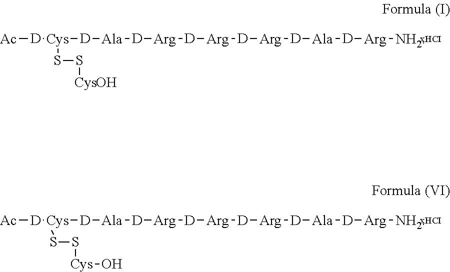

### FIELD OF THE INVENTION

The present invention relates to an improved process for the preparation of Etelcalcetide hydrochloride of Formula (I).

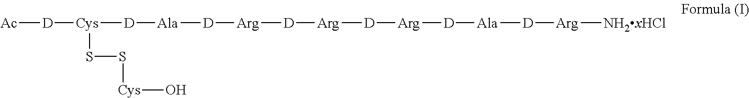

### BACKGROUND OF THE INVENTION

Etelcalcetide is a synthetic peptide calcium-sensing receptor agonist. Etelcalcetide is 8 amino acid peptide with the following Chemical name: N-acetyl-D-cysteinyl-S-(L-cysteine disulfide)-D-alanyl-D-arginyl-D-arginyl-D-arginyl-D-alanyl-D-argininamide and this can be structurally represented as follows:

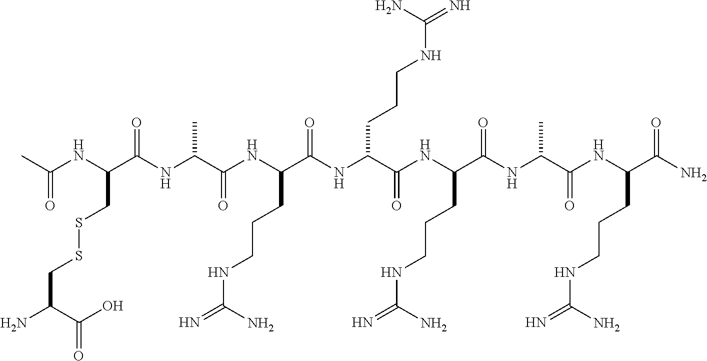

This drug was approved as its hydrochloride salt. The Etelcalcetide hydrochloride of Formula (I) is shown below:

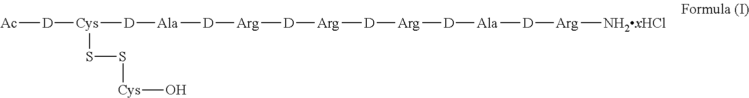

Etelcalcetide is approved in the United States under the trade name PARSABIV® for the treatment of Secondary hyperparathyroidism (HPT) in adult patients with chronic kidney disease (CKD) on hemodialysis.

Etelcalcetide is first described in U.S. Pat. No. 8,377,880. The US '880 patent discloses a process to prepare peptides and conjugates by solid-phase chemistry at 0.25 mmol scale on an ABI automated synthesizer. Sequential coupling of Fmoc-amino acids (4 eq, Anaspec) to Rink-amide resin (Novabiochem) was accomplished using HBTU/DIEA activation. The assembled peptide was cleaved with a TFA cocktail (phenol (5%), triisopropylsilane (2.5%) and water (2.5%); 10 mL per gram of resin) and isolated by precipitation with diethyl ether. After purification using C18 HPLC the final product was isolated in the TFA salt form by lyophilization of appropriate fractions and characterized by HPLC (>95% purity) and LC-MS (confirmed MW).

US Patent application US 2017/0190739 involves conversion of Etelcalcetide TFA salt to Etelcalcetide hydrochloride salt by the addition of 12M aqueous hydrochloric acid. Use of such a high concentrated hydrochloric acid may hydrolyze the peptide resulting in formation of undesired impurities.

US Patent application US 2018/0079777 & Chinese Patent application CN 107434820 uses Iodine, dimethyl sulfoxide & hydrogen peroxide, a conventional method of disulphide bond formation leads to the formation of Etelcalcetide dimers, Cystine and other linear impurities.

However, the processes disclosed in the said documents suffer one or the other problems such as an overall low yield due to formation of high levels of impurities such as Etelcalcetide dimers, Cystine impurities, and other linear impurities.

Therefore, there is a need to develop an improved process for the preparation of Etelcalcetide, which is simple, cost-effective, high purity and high yield, avoids/reduces content of impurities, makes the process robust and particularly one appropriate for commercial scale manufacturing.

### OBJECTIVE OF THE INVENTION

The main objective of the present invention is to provide an improved process for the preparation of Etelcalcetide hydrochloride of Formula (I) by using novel compounds of formula (IV) and (V).

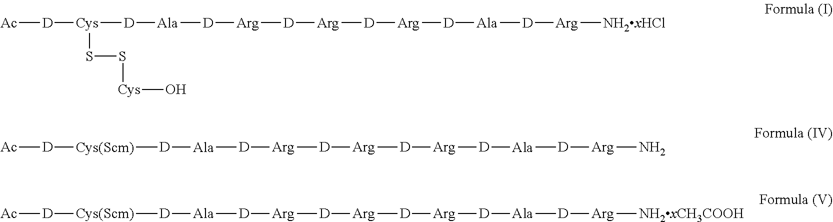

Another objective of the present invention is to provide purification of solution of compound of formula (V) by concentrating the solution using Nano filtration with membrane.

### SUMMARY OF THE INVENTION

In one aspect, the present invention provides a process for preparing Etelcalcetide hydrochloride of Formula (I),

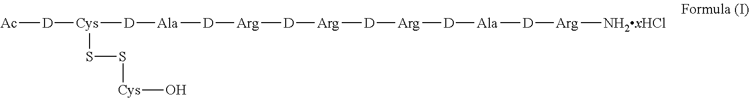

which comprises:

a) Global de-protecting of compound of Formula (II) to form compound of Formula (III);

Ac-D-Cys(X)-D-Ala-D-Arg(pbf)-D-Arg(pbf)-D-Arg(pbf)-D-Ala-D-Arg(pbf)-NH-Resin Formula (II)

Ac-D-Cys(X)-D-Ala-D-Arg-D-Arg-D-Arg-D-Ala-D-Arg-NH₂ Formula (III)

Wherein, X is H, side chain protecting groups;

b) Reacting compound of Formula (III) with methoxycarbonylsulfenyl chloride (Scm) to form crude compound of Formula (IV);

Ac-D-Cys(Scm)-D-Ala-D-Arg-D-Arg-D-Arg-D-Ala-D-Arg-NH₂ Formula (IV)

c) Purifying crude compound of Formula (IV) to form compound of Formula (V), followed by contacted with L-Cysteine to obtain Etelcalcetide acetate of Formula (VI);

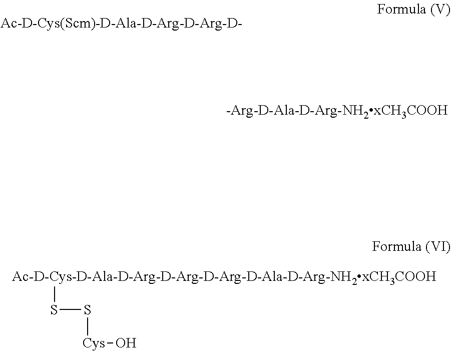

d) Converting Etelcalcetide acetate of Formula (VI) to Etelcalcetide hydrochloride of Formula (I).

In another aspect, the present invention provides a process for preparing Etelcalcetide acetate of Formula (VI), which comprises purifying crude compound of Formula (IV) to form compound of Formula (V), followed by concentrating solution containing compound of Formula (V) by Nano filtration using membrane and contacted with L-Cysteine to obtain Etelcalcetide acetate of Formula (VI).

In further aspect, the present invention provides a process for preparing Etelcalcetide hydrochloride of Formula (I);

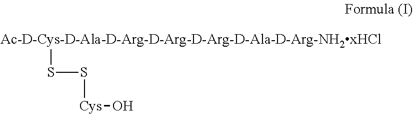

which comprises;

a) Reacting linear peptide of Formula (IIIa)

Ac-D-Cys-D-Ala-D-Arg-D-Arg-D-Arg-D-Ala-D-Arg-NH₂ Formula (IIIa)

with H-Cys(Scm)-OH. TFA to form crude Etelcalcetide.

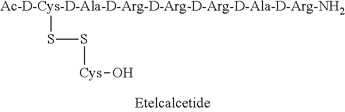

b) Purifying crude Etelcalcetide on preparative HPLC, followed by salt exchange to obtain Etelcalcetide hydrochloride of Formula (I).

### BRIEF DESCRIPTION OF ABBREVIATIONS AND DEFINITIONS

BRIEF DESCRIPTION OF ABBREVIATIONS
AND DEFININTIONS

| | |
|---|---|
| TFA | Trifluoroacetic acid |
| TIPS | Triisopropylsilane |
| DTT | Dithiothreitol |
| TIS | Triisopropylsilane |
| SPPS | Solid phase peptide synthesis |
| EDT | 1,2-ethanedithiol |
| DMS | Dimethyl sulfide |
| IPA | Isopropyl alcohol |
| DMF | N,N-Dimethylformamide |
| HOBt | Hydroxy Benzotriazole |
| MTBE | Methyl-tert.butyl ether |
| Fmoc | Fluorenylmethyloxycarbonyl |

### DETAILED DESCRIPTION OF THE INVENTION

The present invention relates to a process for the preparation of Etelcalcetide Hydrochloride.

In one embodiment, the present invention provides a process for preparing Etelcalcetide hydrochloride of Formula (I);

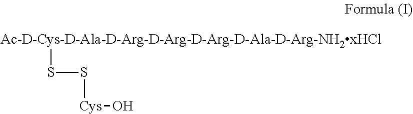

which comprises:

a) Global de-protecting of compound of Formula (II) to form a compound of Formula (III);

Ac-D-Cys(X)-D-Ala-D-Arg(pbf)-D-Arg(pbf)-D-Arg(pbf)-D-Ala-D-Arg(pbf)-NH-Resin Formula (II)

Ac-D-Cys(X)-D-Ala-D-Arg-D-Arg-D-Arg-D-Ala-D-Arg-NH₂ Formula (III)

Wherein, X is H, side chain protecting groups;

b) Reacting compound of Formula (III) with methoxycarbonylsulfenyl chloride (Scm) to form crude compound of Formula (IV);

Ac-D-Cys(Scm)-D-Ala-D-Arg-D-Arg-D-Arg-D-Ala-D-Arg-NH₂ Formula (IV)

c) Purifying crude compound of Formula (IV) to form compound of Formula (V), followed by contacted with L-Cysteine to obtain Etelcalcetide acetate of Formula (VI);

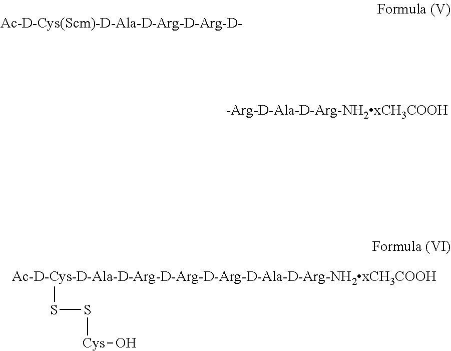

d) Converting Etelcalcetide acetate of Formula (VI) to Etelcalcetide hydrochloride of Formula (I).

Compound of Formula (II) can be obtained according to the processes known in the art.

The side chain protecting groups (X) used in step a) is selected from the group consisting of acetamidomethyl (Acm), trityl (Trt), benzyl (Bzl), tert-butyl (tBu), tert-butylthio (tButhio), p-methoxybenzyl (pMeoBzl), Phenylacetamidomethyl (Phacm), 4-methyltrityl (Mtt) and 4-methoxytrityl (Mmt). Preferably acetamidomethyl (Acm), trityl (Trt) and Benzyl (Bzl).

The global de-protection is performed in presence of cocktail mixture selected from TFA, TIPS, DTT, TIS, EDT, DMS, thioanisole, phenol, anisole or mixture thereof. Preferably TFA:TIPS:DTT:solvent (or) TFA:TIPS:DTT:water:solvent (or) TFA:TIS:solvent. Wherein, the solvent is comprising water, dimethyl sulfide, methanol, ethanol, 1-propanaol, isopropanol, n-butanol, dichloromethane, dichloroethane, chlorobenzene, diethyl ester, tetrahydrofuran, diisopropyl ether or mixture thereof. Preferably TFA:TIS:Water:DTT:Phenol.

Linear peptide compound of Formula (III) reacts with Methoxycarbonylsulfenyl chloride (Scm) in the presence of trifluoroacetic acid followed by isolating crude peptide compound of formula (IV) by precipitating with pre-cool MTBE, followed by dried under vacuum.

Further, purifying crude compound of Formula (IV) on preparative HPLC is performed with buffer system comprising 0.5% acetic acid as buffer A and 100% acetonitrile as buffer B to form compound of Formula (V) and subjecting the solution of compound of Formula (V) to Nano filtration using 300 D molecular weight cut-off membrane and concentration up to ⅕^{th} volume (from original volume) and added equal amount of water to the retentate and concentrate of 20 grams/Liter as final concentration. The concentrated solution is contacted with L-cysteine to obtain Etelcalcetide acetate of Formula (VI).

L-cysteine is selected from L-cysteine hydrochloride and L-cysteine hydrochloride monohydrate.

Etelcalcetide acetate of Formula (VI) is loaded on preparative HPLC, column packed with reverse phase media (C18). De-salting was done by passing 3 void volume of 0.1M ammonium chloride in purified water fallowed by elution of product from the column by using very dilute HCl in purified water. The fractions collected and purity of fractions are monitored by analytical HPLC.

The fractions containing pure Etelcalcetide hydrochloride (>98.5%) are pooled and filtered through 0.2 micron filter. The resulting peptide solution is freeze-dried to isolate Etelcalcetide hydrochloride of Formula (I).

In another embodiment, the present invention provides a process for preparing Etelcalcetide acetate of Formula (VI), which comprises purifying crude compound of Formula (IV) to form compound of Formula (V), followed by concentrating solution containing compound of Formula (V) by Nano filtration using membrane and contacted with L-Cysteine to obtain Etelcalcetide acetate of Formula (VI);

Purifying crude compound of Formula (IV) on preparative HPLC is performed with buffer system comprising 0.5% acetic acid as buffer A and 100% acetonitrile as buffer B to form compound of Formula (V) and subjecting the solution of compound of formula (V) to Nano filtration using 300 D molecular weight cut-off membrane and concentration up to ⅕^{th} volume (from original volume) and added equal amount of water to the retentate and concentrate of 20 grams/Liter as final concentration. The concentrated solution is contacted with L-cysteine to obtain Etelcalcetide acetate of Formula (VI).

L-cysteine used is in the form of L-cysteine hydrochloride and L-cysteine hydrochloride monohydrate.

In further embodiment, the present invention provides a process for preparing Etelcalcetide hydrochloride of Formula (I);

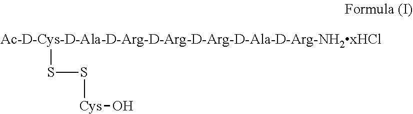

Which comprises;

a) Reacting linear peptide of Formula (IIIa)

Ac-D-Cys-D-Ala-D-Arg-D-Arg-D-Arg-D-Ala-D-Arg-NH₂ Formula (IIIa)

with H-Cys(Scm)-OH. TFA to form crude Etelcalcetide.

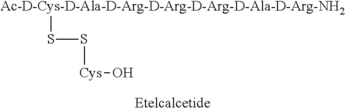

b) Purifying crude Etelcalcetide on preparative HPLC, followed by salt exchange to obtain Etelcalcetide hydrochloride of Formula (I).

Linear peptide compound of formula (III) is dissolved in degassed aqueous methanol at a concentration of 1 gram/100 ml. After dissolution of H-Cys(Scm)-OH.TFA is added and stirred for 1 hour. Progress of reaction was monitored by analytical reverse phase HPLC & Ellman's test. After completion of reaction the obtained crude Etelcalcetide was filtered through 2.4 micron filter and used as such for next stage purification.

Etelcalcetide (crude) is purified on preparative HPLC, column packed with reverse phase media using gradient method, where buffer is 0.5% acetic acid/ammonium acetate and 100% acetonitrile (as buffer B). The fractions are collected and purity of fractions monitored by analytical HPLC. Fractions containing >95% pure are pooled as main pool; and fractions not meeting the pooling criteria re-processed in a similar manner.

The main pool is diluted with equal amount of purified water or organic modifier is removed under vacuum and thereafter loaded on preparative HPLC, column packed with polymeric reverse phase media.

De-salting is done by passing 3 void volume of 0.1M ammonium chloride in purified water followed by elution of product from the column by using very dilute HCl in purified water.

The fractions are collected, and purity of fractions monitored by analytical HPLC.

The fractions containing pure Etelcalcetide hydrochloride (>98.5%) are pooled and filtered through 0.2 micron filter. The resulting peptide solution freeze-dried to isolate Etelcalcetide hydrochloride of Formula (I).

Having described the invention with reference to certain aspects and embodiments, which will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### Example-1: Synthesis of Ac-D-Cys(Trt)-D-Ala-D-Arg(pbf)-D-Arg(pbf)-D-Arg(pbf)-D-Ala-D-Arg(pbf)-NH-Resin

### Step A

Rink amide AM Resin 1.21 kg, (substitution 0.66 mill mole/gram) was taken in a 20 L SPPS reactor, 12.1 L of DMF was added and allowed it to swell for 20 minutes and drained.

### Step B

The above resin was de-blocked with one bed volume of 20% piperidine in DMF (twice) for 5 minutes and 20 minutes and washed with one bed volume of DMF (2 times), IPA (2 times) and DMF (2 times).

### Step C

Fmoc-D-Arg(pbf)-OH (780.0 grams, 1.5 equivalents) and HOBT.H₂O (184.0 grams, 1.5 equivalents) were dissolved in DMF (6.0 L), and Diisopropylcarbodiimide (308 mL, 2.5 equivalents) was added and stirred the reaction mixture for 3 minutes. It was added to the resin in Step A and stirred for two hours at room temperature. The progress of the coupling was monitored by Kaiser Test. After completion of the reaction, the resin was drained and washed with one bed volume DMF for 5 minutes and drained.

Encapping: A solution of Diisopropylethylamine (420.0 mL, 3 equivalents) was prepared in dichloromethane (6.0 L), acetic anhydride (228.0 mL, 3 equivalents) and added to the resin. It was stirred for 30 minutes and drained.

Washed the resin with

a) one bed volume DMF for 3 minutes (2 times) and drained.

b) one bed volume IPA for 3 minutes (2 times) and drained.

c) one bed volume DMF for 3 minutes (2 times) and drained.

The above resin was deblocked with one bed volume of 20% piperidine in DMF (twice) for 5 minutes and 20 minutes and washed with one bed volume of DMF (2 times), IPA (2 times) and DMF (2 times).

### Step D

Fmoc-D-Ala-OH (500.0 grams, 2 equivalents) and HOBT.H₂0 (248.0 grams, 2 equivalents) were dissolved in DMF (6.0 L) and while stirring DIC (372.0 mL, 3 equivalents) was added and stirred the reaction mixture for 3 minutes. It was added to the resin and stirred for two to three hours at room temperature. The progress of coupling was monitored by Kaiser Tests. After completion of the reaction the resin was drained and washed with one bed volume of DMF.

The repeated cycles of operations (Fmoc Deprotections and Amino acid couplings) were performed sequentially for Fmoc-D-Arg(pbf)-OH, Fmoc-D-Arg(pbf)-OH, Fmoc-D-Arg(pbf)-OH, Fmoc-D-Ala-OH and Fmoc-D-Cys(Trt)-OH, to obtain H-D-Cys(Trt)-D-Ala-D-Arg(pbf)-D-Arg(pbf)-D-Arg(pbf)-D-Ala-D-Arg(pbf)-NH-Resin

### Step E

### N-Acetylation:

A solution of acetic anhydride (228.0 mL, 3 equivalents) was prepared in DMF (8.48 L) and added to the resin. It was stirred for 30 minutes and drained. Prepared a solution of acetic anhydride (228.0 mL, 3 equivalents) in DMF (8.48 L) and added to the resin. It was stirred for 30 minutes and drained.

The progress of Acetylation was monitored by Kaiser Test. After completion of the reaction the resin was drained and washed with one bed volume of DMF (2 times), one bed volume of IPA (2 times) and one bed volume of MTBE (2 times). Finally the peptide resin was isolated and dried to obtained Ac-D-Cys(Trt)-D-Ala-D-Arg(pbf)-D-Arg(pbf)-D-Arg(pbf)-D-Ala-D-Arg(pbf)-NH-Resin;

Peptidyl resin: 3.2 kg.

### Example-2: Preparation of Ac-D-Cys-D-Ala-D-Arg-D-Arg-D-Arg-D-Ala-D-Arg-NH₂ (Linear Peptide)

Deblocking of protected peptide was performed with a Cocktail of TFA+TIS+Water+DTT+Phenol (85%+5%+2.5%+2.5+5) for 3 hours at room temperature. The crude peptide was isolated by precipitating with pre-cool MTBE and dried under vacuum to obtain linear peptide.

Weight: 808.0 grams, Yield: 96.8%, Purity by HPLC: 58.32%.

### Example-3: Preparation and Purification of Ac-D-Cys(Scm)-D-Ala-D-Arg-D-Arg-D-Arg-D-Ala-D-Arg-NH₂

Linear peptide (806.0 grams) obtained from Example-2 was treated with methoxycarbonyl sulfenyl chloride (88.0 mL, 1.1 equivalents) in Trifluoroacetic acid for half an hour, completion of reaction was monitored by HPLC.

The crude peptide was isolated by precipitating with pre-cool MTBE and dried under vacuum to obtain linear (scm) peptide (crude) 1.1 kg.

Crude obtained was purified on preparative HPLC, column packed with reverse phase media (C18) using gradient method, where buffer A is 0.5% Acetic acid and 100% acetonitrile as buffer B.

The fractions were collected, and purity of fractions were monitored by analytical HPLC.

Fractions containing >95% pure were pooled as main pool; and fractions not meeting the pooling criteria were re-processed in a similar manner 766.0 grams of compound present in Main Pool.

Yield: 84.2%, Purity by HPLC: 98.48%.

### Example-4: Preparation of Etelcalcetide Acetate

The solution obtained from Example-3 was subjected to Nano filtration using 300 Daltons molecular weight cut-off membrane and concentration up to ⅕^{th} volume (from original volume). Add equal amount of water to the retentate and concentrate of 20 gram/Liter as final concentration.

The concentrated solution was treated with cysteine for 30 minutes to obtain Etelcalcetide as acetate salt, Purity by HPLC: 93.67%.

### Example-5: Salt Exchange and Lyophilization

The solution obtained from Example-4 were loaded on preparative HPLC, column packed with reverse phase media (C18). De-salting was done by passing 3 void volume of 0.1 M ammonium chloride in purified water fallowed by elution of product from the column by using very dilute HCl in purified water. The fractions were collected and purity of fractions were monitored by analytical HPLC. The fractions containing pure Etelcalcetide hydrochloride (>98.5%) were pooled and filtered through 0.2 micron filter. The resulting peptide solution was freeze-dried to isolate Etelcalcetide as hydrochloride salt. Practical weight: 252 grams;

Yield: 30.07%; Purity by HPLC: 99.70%; Mass: 1047.5 Dalton's.

### Example-6: Preparation of Crude Etelcalcetide

Linear peptide obtained from Example-2 was dissolved in degassed aqueous methanol at a concentration of 1 gram/100 ml. After dissolution of H-Cys(Scm)-OH.TFA (2 equivalents) was added and stirred for 1 hour. Progress of reaction was monitored by analytical reverse phase HPLC & Elman's test, after completion of reaction the obtained crude Etelcalcetide was filtered through 2.4 micron filter and used as such for next stage purification.

### Example-7: Purification of Crude Etelcalcetide

Crude Etelcalcetide solution obtained from Example-6 was purified on preparative HPLC, column packed with polymeric reverse phase media using gradient method, where buffer is 0.5% Acetic acid/Ammonium acetate and 100% acetonitrile (as buffer B). The fractions were collected and purity of fractions were monitored by analytical HPLC. Fractions containing >95% pure were pooled as main pool; and fractions not meeting the pooling criteria were re-processed in a similar manner.

### Example-8: Salt Exchange and Lyophilization

The main pool obtained from Example-7 were diluted with equal amount of purified water or organic modifier was removed under vacuum and thereafter loaded on preparative HPLC, column packed with reverse phase media.

De-salting was done by passing 3 void volume of 0.1 M ammonium chloride in purified water fallowed by elution of product from the column by using very dilute HCl in purified water.

The fractions were collected and purity of fractions were monitored by analytical HPLC.

The fractions containing pure Etelcalcetide hydrochloride (>98.5%) were pooled and filtered through 0.2 micron filter. The resulting peptide solution was freeze-dried to isolate Etelcalcetide as hydrochloride salt.

We claim:

1. A process for preparation of Etelcalcetide hydrochloride of Formula (I); 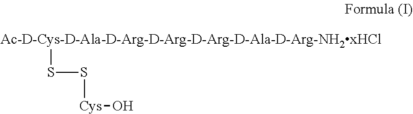
Which comprises:
(a) Global de-protecting of compound of Formula (II) to obtain a compound of Formula (III);
Ac-D-Cys(X)-D-Ala-D-Arg(pbf)-D-Arg(pbf)-D-Arg(pbf)-D-Ala-D-Arg(pbf)-NH-Resin Formula (II)
Ac-D-Cys(X)-D-Ala-D-Arg-D-Arg-D-Arg-D-Ala-D-Arg-NH₂ Formula (III)
Wherein, X is H, side chain protecting groups;
(b) Reacting compound of Formula (III) with methoxycarbonylsulfenyl chloride (Scm) in the presence trifluoroacetic acid to obtain crude compound of Formula (IV);
Ac-D-Cys(Scm)-D-Ala-D-Arg-D-Arg-D-Arg-D-Ala-D-Arg-NH₂ Formula (IV)
c) Purifying crude compound of Formula (IV) to obtain compound of Formula (V), followed by contacted with L-Cysteine to obtain Etelcalcetide acetate of Formula (VI); 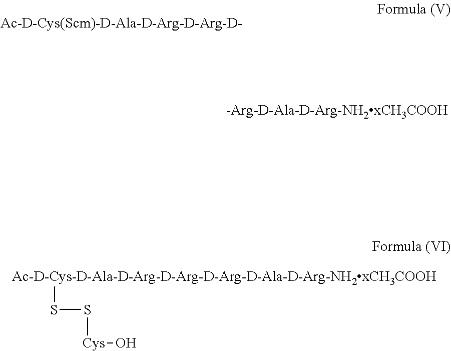
d) Converting Etelcalcetide acetate of Formula (VI) to Etelcalcetide hydrochloride of Formula (I).

2. The process as claimed in claim 1, wherein, the side chain protecting groups (X) used in step (a) is selected from the group comprises Acm, Trt, Bzl, tBu, tButhio, pMeoBzl, Phacm, Mtt or Mmt.

3. The process as claimed in claim 1, wherein the global de-protection using cocktail mixture selected from TFA, TIPS, DTT, TIS, EDT, DMS, thioanisole, phenol, anisole or mixture thereof.

4. The process as claimed in claim 3, wherein the global de-protection using cocktail mixture selected from TFA:TIPS:DTT:solvent (or) TFA:TIPS:DTT:water:solvent (or) TFA:TIS:solvent (or) EDT, DMS, thioanisole, phenol, anisole or mixture thereof.

5. The process as claimed in claim 4, wherein the solvent selected from water, dimethyl sulfide, methanol, ethanol, 1-propanaol, isopropanol, n-butanol, dichloromethane, dichloroethane, chlorobenzene, diethyl ester, tetrahydrofuran, diisopropyl ether or mixture thereof.

6. The process as claimed in claim 1, wherein step (c) is performed on preparative HPLC with buffer system comprises 0.5% acetic acid as buffer A and 100% acetonitrile as buffer B.

7. The process as claimed in claim 1, wherein L-cysteine used in step (c) is in the form of L-cysteine hydrochloride and L-cysteine hydrochloride monohydrate.

8. A process for preparation of Etelcalcetide acetate of Formula (VI); 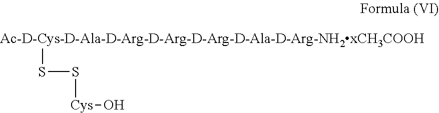
Which comprises:
a) purifying crude compound of Formula (IV) with 0.5% acetic acid (buffer A) and 100% acetonitrile (buffer B) to obtain compound of Formula (V);
Ac-D-Cys(Scm)-D-Ala-D-Arg-D-Arg-D-Arg-D-Ala-D-Arg-NH₂ Formula (IV)
Ac-D-Cys(Scm)-D-Ala-D-Arg-D-Arg-D-Arg-D-Ala-D-Arg-NH₂.xCH₃COOH Formula (V)
b) concentrating solution containing compound of Formula (V) by Nano filtration using membrane;
c) contacting the compound of Formula (V) with L-Cysteine to obtain Etelcalcetide acetate of Formula (VI).

9. A process for preparation of Etelcalcetide hydrochloride of Formula (I); 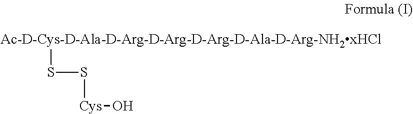
Which comprises:
a) Reacting linear peptide of Formula (IIIa)
Ac-D-Cys-D-Ala-D-Arg-D-Arg-D-Arg-D-Ala-D-Arg-NH₂ Formula (IIIa)
with H-Cys(Scm)-OH. TFA to form crude Etelcalcetide; 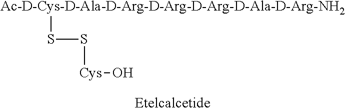
b) Purifying crude Etelcalcetide with 0.5% acetic acid or ammonium acetate (buffer A) and 100% acetonitrile (buffer B), followed by salt exchange to obtain Etelcalcetide hydrochloride of Formula (I).
